(19) United States
(12) Patent Application Publication
(10) Pub. No.: US 2004/0253211 A1
(43) Pub. Date: Dec. 16, 2004

(54) Method for diagnosing and treating cancer

(75) Inventors: Steven C. Quay, Edmonds, WA (US); Kunyuan Cui, Bothell, WA (US)

Correspondence Address: Paul G. Lunn, Nastech Pharmaceutical Company Inc., 3450 Monte Villa Parkway, Bothell, VA, 98021-8906, US

(73) Assignee: Nastech Pharmaceutical Company Inc.

(21) Appl. No.: 10/836,421

(22) Filed: Apr. 30, 2004

Related U.S. Application Data
(60) Provisional application No. 60/466,905, filed on Apr. 30, 2003.

(51) Int. Cl. A61K 48/00; C12N 15/86

(52) U.S. Cl. 424/93.2; 514/44; 435/456

(57) ABSTRACT

A method for treating cancer in a mammal comprised of administering a therapeutically effective amount of an agent that induces the expression of one or more claudins within a cancerous cell. Preferably the claudin is claudin-3, claudin-4 aor claudin-9. In a preferred method, a nucleic acid that encodes a claudin is administered to the mammal under conditions wherein the nucleic acid is transfected into the cancerous cell and the claudin is produced in the cell. Also disclosed is a method for diagnosing cancer comprising determining the presence of a claudin 3, 4 or 9 in a cell that normally expresses the protein. If the cell does not express the claudin, then it is cancerous.

### CROSS-REFERENCE TO RELATED APPLICATIONS

This claims priority under 35 U.S.C. §119(e) of U.S. Provisional Application No. 60/466,905 filed on Apr. 30, 2003, the contents of which are incorporated herein by reference.

### BACKGROUND OF THE INVENTION

Cancers develop from uncontrolled multiplication of cells. All cancers are life threatening. Even when cancer does not result in death, it is debilitating, not only to the patient, but also to family, friends and co-workers. Too often, moreover, cancers prove fatal. The personal and public loss from this cluster of diseases, which cause a significant fraction of all premature deaths, is beyond estimation.

Although effective treatment modalities have been developed in a few cases, many cancers remain refractory to currently available therapies. Particularly difficult to treat are metastatic cancers. These cancers pose the highest risk to patients and, for optimal prognosis, often must be treated by aggressive methods that present increased risks of deleterious side-effects. Therefore, there is a great need for methods that accurately distinguish those tumors that are likely to metastasize from those that are unlikely to do so. Furthermore, methods for treating metastatic cancers often are inadequate, and there also is a clear need for improved anti-metastatic agents and methods to treat metastatic cancers.

Metastatic cancers originate from a primary tumor. Metastasis of the primary tumor produces secondary tumors and disseminated cancer. It is well known that both primary and secondary tumors shed large numbers of cells. The shed cells can spread through the body. For instance, a primary tumor may damage the surrounding lymph or circulatory vessels, allowing entry of shed cells into the lymph or circulatory systems, and hastening their spread in the body. Moreover, shedding of cells by cancerous tumors increases during surgery and radiotherapy.

Most shed cells do not form new tumors. To do so such cells must surmount a series of physical and physiological barriers. In fact, a series of distinct events must occur for metastasis to occur. The primary tumor physically must invade interstitial space of the primary tissue. In particular, it must penetrate the basement membrane of the tissue. For most metastases the tumor must damage the endothelial cell wall of lymphatic or vascular vessels to provide access to shed cells. Cells that enter the lymph or blood must survive hemodynamic stress and host defenses in the circulation and, furthermore, the cells must lodge at a new site in the circulatory system, a process that apparently involves aggregated platelets. A cell then must extravasate out of the vessel into the interstitial space. Finally, it must invade the interstitial space of the secondary organ and proliferate in the new location. Although the process of metastasis is physiologically complex, the overall pattern of metastasis is general to many types of cancers.

The metastatic process also clearly involves complex intracellular mechanisms that alter cancerous cells and their interactions with surrounding cells and tissues. For instance, cancerous cells are characterized by aberrant expression of adhesion proteins, enzymes that degrade matrix components, autocrine factors, ligand-responsive receptors, factors of angiogenesis and prostaglandins, to name a few. In particular, the signaling pathways that initiate tumor cell migration are among the least understood aspects of invasion and metastasis. Currently, it is thought that proliferation of many cancerous cells depends upon specific ligand-receptor interactions. Thus far, however, it has not been possible to use this paradigm, or other concepts of the underlying mechanisms of metastasis, to develop a therapy that prevents or effectively inhibits metastasis of metastatic cancers.

The complexity of the processes involved in metastasis, and the lack of understanding of underlying molecular mechanisms, have made it particularly difficult, in some cases, to distinguish tumors that are likely to metastasize from those that are unlikely to do so. The inability to discern the metastatic potential of tumors precludes accurate prognosis and leads, inevitably, to the therapeutic intervention that either is too aggressive or insufficiently aggressive. Clinically it is also important to determine if a tumor is metastatic in deciding whether or not to administer chemotherapy or limit the treatment to surgical removal of the tumor. Furthermore, for all types of cancers it has been difficult or impossible, thus far, to develop treatments that inhibit or prevent the spread of metastatic tumors. Clearly, there remains a great need for methods to accurately determine the metastatic potential of tumors and for effective anti-metastatic compositions and methods.

### DISCLOSURE OF THE INVENTION

The present invention fills this need by providing for a method for inhibiting the metastasis of tumor cells comprising promoting the expression of one or more of the following claudin proteins, namely claudin-3 (SEQ ID NOs: 1 and 2), claudin-4 (SEQ ID NOs: 3 and 4) and claudin-9 (SEQ ID NOs: 5 and 6). The present invention results from a surprising discovery resulting from studies examining the expression of claudins in normal and transformed cell lines. The results indicate sharp and clear differences in claudins-3, claudin-4 and claudin-9 expression between tissues from normal and transformed cell lines. Claudin 3, 4 and 9 were expressed in normal tissues, but were not expressed in transformed, cancerous cells.

The invention therefore provides in one aspect a method of diagnosis of neoplasia, which method comprises analyzing the expression of the expression of claudin-3, claudin-4 and claudin-9. If these genes are under-expressed, this indicates a transformed/cancerous cell is a metastatic cell. Methods include nucleic acid hybridization technique for detecting the presence of the claudins including RT-PCR methods and kits. Also claimed are diagnostic kits such in vitro PCR assay kit for a first container comprising PCR primers that amplify said claudin transcript or cDNA generated therefrom; and a second container comprising a nucleic acid marker.

Also claimed are antibody methods and kits for detecting the presence of metastatic cancer. The kit is comprised of a polypeptide, protein, a antibody or antibody fragment that specifically binds to a mammalian a claudin-3, claudin-4 or claudin-9 polypeptide. Included are enzyme-linked immunosorbent assay, radioimmunoassay, and fluorescent immunoassays.

The diagnosis of neoplasia may refer to the initial detection of neoplastic tissue or it may be the step of distinguishing between metastatic and non-metastatic tumors. References to the term “diagnosis” as used herein are to be understood accordingly.

The method is particularly applicable to the diagnosis of solid tumors particularly malignant tumors e.g. carcinomas. The sample on which the assay is performed is preferably of body tissue or body fluid, and not of cells cultured in vitro. The sample may be a small piece of tissue or a fine needle aspirate (FNA) of cells from a solid tumor. Alternatively, it may be a sample of blood or urine or another body fluid, a cervical scraping or a non-invasively obtained sample such as sputum, urine or stool.

The cDNA may be detected by use of one or more labeled specific oligonucleotide probes, the probes being chosen so as to be capable of annealing to part of the amplified cDNA sequence. Alternatively, labeled oligonucleotide primers and/or labeled mononucleotides could be used. There are a number of suitable detectable labels, which can be employed, including radiolabels.

The level of gene expression of claudin-3, claudin-4 and claudin-9 can be determined by RT-PCR, or by using labeled antibodies that bind to claudin-3, claudin-4 or claudin-9. For example, labeled antibodies that bind to claudin-3, claudin-4 or claudin-9 can be used to stain tissues expressing the proteins. If the tissues normally express the claudins but the antibodies to the claudins do not bind to the surface of the tissue as indicated by the lack of production of the desired stain or other label, this indicates that the claudin is not expressed by the tissue and that the tissue is cancerous and metastatic.

The present invention is further directed to a method for treating a metastatic cancer comprising inducing the expression of claudin-3, claudin-4 or claudin-9. A preferred embodiment is comprised of transfecting a cancerous cell with a nucleic acid encoding a claudin-3 (SEQ ID NOs: 5 and 6), claudin-4 (SEQ ID NOs: 7 and 8) or claudin-9 (SEQ ID NOs: 17 and 18). Preferably, the nucleic acid is a DNA contained within a suitable vector such as an adenoviral vector (See U.S. Pat. No. 5,547,932), an adenovirus-associated virus vector (See U.S. Pat. No. 6,541,258; U.S. Pat. No. 5,658,776 and U.S. Pat. No. 6,346,415) or a suitable retroviral vector (See U.S. Pat. No. 5,736,387).

A cancer is a general term used to indicate any of various types of malignant neoplasms, most of which invade surrounding tissues, may metastasize to several sites, and are likely to recur after attempted removal and to cause death the patient unless adequately treated. A cancer includes both sarcomas and carcinomas.

A metastatic cancer is a cancer that can spread to other parts of the body.

Human claudin-3 is a polypeptide and has the following 220 amino acid residues: MSMGLEITGTALAVLGWLGTIVCCALPMWRVSAFIGSNIITSQNIWEGLWM NCVVQSTGQMQCKVYDSLLALPQDLQAARALIVVAILLAAFGLLVALVGA QCTNCVQDDTAKAKITIVAGVLFLLAALLTLVPVSWSANTIIRDFYNPWPEA QKREMGAGLYVGWAAAALQLLGGALLCCSCPPREKKYTATKWYSAPRST GPGASLGTGYDRKDYV (SEQ ID NO: 2), the underlined portion of which is the extracellular domain.

Human claudin-4 is a polypeptide of 209 amino acids.

MASMGLQVMGIALAVLGWLAVMLCCALPMWRVTAFIGSNIVTSQTIWEGL WMNCWQSTGQMOCKVYDSLLALPQDLQAARALVIISIIVAALGVLLSW GGKCTNCLEDESAKAKTMIVAGVVFLLAGLMVIVPVSWTAHNIIQDFYNPLV ASGQKREMGASLYVGWAASGLLLLGGGLLCCNCPPRTDKPYSAKYSAARS AAASNYV (SEQ ID NO: 4). The extracellular domain of the amino acid sequence is underlined.

Human claudin-9 is a polypeptide of 217 amino acids.

MASTGLELLGMTLAVLGWLGTLVSCALPLWKVTAFIGNSIWVAQVVWEGL WMSCWQSTGOMQCKVYDSLLALPQDLQAARALCVIALLLALLGLLVAIT GAQCTTCVEDEGAKARIVLTAGVILLLAGILVLIPVCWTAHAIIQDFYNPLVA EALKRELGASLYLGWAAAALLMLGGGLLCCTCPPPQVERPRGPRLGYSIPSR SGASGLDKRDYV (SEQ ID NO: 6). The extracellular domain of the amino acid sequence being underlined.

The tight junction (TJ) of epithelial and endothelial cells is a particularly important cell-cell junction that regulates permeability of the paracellular pathway, and also divides the cell surface into apical and basolateral compartments. Tight junctions form continuous circumferential intercellular contacts between epithelial cells and create a regulated barrier to the paracellular movement of water, solutes, and immune cells. They also provide a second type of barrier that contributes to cell polarity by limiting exchange of membrane lipids between the apical and basolateral membrane domains.

Tight junctions are thought to be directly involved in barrier and fence functions of epithelial cells by creating an intercellular seal to generate a primary barrier against the diffusion of solutes through the paracellular pathway, and by acting as a boundary between the apical and basolateral plasma membrane domains to create and maintain cell polarity, respectively. Tight junctions are also implicated in the transmigration of leukocytes to reach inflammatory sites. In response to chemoattractants, leukocytes emigrate from the blood by crossing the endothelium and, in the case of mucosal infections, cross the inflamed epithelium. Transmigration occurs primarily along the paracellular rout and appears to be regulated via opening and closing of tight junctions in a highly coordinated and reversible manner. On ultrathin section electron micrographs, TJs appear as a set of discrete sites of apparent fusion involving the outer leaflet of plasma membranes of adjacent cells. On freeze-fracture electron micrographs of most epithelial cells, TJs appear as a set of continuous, anastomosing intramembranous particle strands (TJ strands) in the protoplasmic face (P-face) with complementary grooves in the extracellular (E)-face.

Numerous proteins have been identified in association with TJs, including both integral and peripheral plasma membrane proteins. Current understanding of the complex structure and interactive functions of these proteins remains limited. Among the many proteins associated with epithelial junctions, several categories of trans-epithelial membrane proteins have been identified that may function in the physiological regulation of epithelial junctions. These include a number of “junctional adhesion molecules” (JAMs) and other TJ-associated molecules designated as occludins, claudins, and zonulin.

JAMs, occludins, and claudins extend into the paracellular space, and these proteins in particular have been contemplated as candidates for creating an epithelial barrier between adjacent epithelial cells and regulatable channels through epithelial cell layers. In one model, occludin, claudin, and JAM have been proposed to interact as homophilic binding partners to create a regulated barrier to paracellular movement of water, solutes, and immune cells between epithelial cells. The mechanism of the present invention is based upon the discovery that metastatic cancer cells have lost their ability to form tight junctions between other cells, and thus the cells are free to metastasize throughout the body.

The DNA encoding a claudin to be used in the process of the present invention should encode at least the extracellular domain of human claudin-3, claudin-4, or claudin-9, and a domain capable of binding to the cell membrane or a transmembrane domain, particularly those of claudin-3, claudin-4 and claudin-9. It is believed that the proteins expressed by these claudins inhibit the metastasis of the cancerous cells. If claudin-3, claudin-4 and claudin-9 are then induced to be expressed, the cancerous cells may be induced to differentiate into normal cells or at least cells that can again form tight junctions, thus losing their ability to metastasize.

This invention provides a method for reversing the cancerous phenotype of a cancer cell, which comprises introducing a nucleic acid comprising a claudin gene into the cell under conditions permitting the expression of the gene so as to thereby reverse the cancerous phenotype of the cell.

This invention also provides a method for reversing the cancerous phenotype of a cancer cell in a subject, which comprises introducing a nucleic acid molecule comprising a claudin gene into the subject's cancerous cell under conditions permitting expression of the gene in the subject's cells so as to thereby reverse the cancerous phenotype of the cell.

Methods to introduce a nucleic acid molecule into cells have been well known in the art. Naked nucleic acid molecule may be introduced into the cell by direct transformation. Alternatively, the nucleic acid molecule may be embedded in liposomes. Accordingly, this invention provides the above methods wherein the nucleic acid is introduced into the cells by naked DNA technology, adenovirus vector, adenovirus-associated virus vector, Epstein-Barr virus vector, Herpes virus vector, attenuated HIV vector, retroviral vectors, vaccinia virus vector, liposomes, antibody-coated liposomes, mechanical or electrical means. The above-recited methods are merely served as examples for feasible means of introduction of the nucleic acid into cells. Other methods known may be also be used in this invention.

In an embodiment of the above methods, the claudin gene is linked to a regulatory element such that its expression is under the control of the regulatory element. In a still further embodiment, the regulatory element is inducible or constitutive. Inducible regulatory element like an inducible promoter is known in the art. Regulatory element such as promoter, which can direct constitutive expression is also known in the art. In a separate embodiment, the regulatory element is a tissue specific regulatory element. The expression of the claudin gene will then be tissue-specific. The invention also provides a pharmaceutical composition, which comprises an amount of a nucleic acid comprising a claudin gene effective to reverse the cancerous phenotype of a cancer cell and a pharmaceutically acceptable carrier.

Accordingly, in a further embodiment of the invention, nucleic acids which encode claudin-3, claudin-4 or claudin-9 of the present invention can be inserted into vectors and used as gene therapy vectors. In one embodiment, the gene therapy vector is a viral vector, e.g. replication defective retroviruses, adenoviruses and adeno-associated viruses, wherein the nucleic acid molecule encoding the photosensitive protein is ligated into the viral genome. Viral vectors, including lentiviral, retroviral, and adeno-associated virus vectors, are generally understood to be the gene therapy vector of choice for the transfer of exogenous genes in vivo, particularly into humans. Examples of lentiviral vectors include, but are not limited to, HIV, FIV, BIV, EIAV, and SIV. Viral vectors provide efficient delivery of genes into cells, and the transferred nucleic acids are stably integrated into the chromosomal DNA of the host. The infectivity of the viral vector can be made cell-specific by expressing cell-specific proteins on the surface of the viral particle which will interact with receptors unique to the cell of interest. In this manner, the viral vector can be targeted to retinal ganglion cells. Expression is further enhanced by the use of tissue- or cell-specific transcriptional regulatory sequences which control expression of the gene. See, Anderson et al., U.S. Pat. No. 5,399,346; Mann et al., Cell, 33:153 (1983); Temin et al., U.S. Pat. No. 4,650,764; Temin et al., U.S. Pat. No. 4,980,289; Markowitz et al., J. Virol., 62:1120 (1988); Temin et al., U.S. Pat. No. 5,124,263; International Patent Publication No. WO 95/07358, published Mar. 16, 1995 by Dougherty et al.; and Blood, 82:845 (1993).

Alternatively, the vector can be introduced by lipofection in vivo using liposomes. Synthetic cationic lipids can be used to prepare liposomes for in vivo transfection of a gene encoding a marker [Felgner et al., Proc. Natl. Acad. Sci. USA, 84:7413-7417 (1987); see Mackey et al., Proc. Natl. Acad. Sci. USA, 85:8027-8031 (1988)]. The use of lipofection to introduce exogenous genes into specific organs in vivo has certain practical advantages. Molecular targeting of liposomes to specific cells represents one area of benefit. It is clear that directing transfection to particular cells represents one area of benefit. It is clear that directing transfection to particular cell types would be particularly advantageous in a tissue with cellular heterogeneity, such as the pancreas, liver, kidney, and brain. Lipids may be chemically coupled to other molecules for the purpose of targeting. Targeted peptides, e.g., hormones or neurotransmitters, and proteins such as antibodies, or non-peptide molecules could be coupled to liposomes chemically.

The step of facilitating the production of infectious viral particles in the cells may be carried out using conventional techniques, such as standard cell culture growth techniques. The step of collecting the infectious virus particles also can be carried out using conventional techniques. For example, the infectious particles can be collected by cell lysis, or collection of the supernatant of the cell culture, as is known in the art. Optionally, the collected virus particles may be purified if desired. Suitable purification techniques are well known to those skilled in the art. Alternatively, the viral vectors of the invention can be administered ex vivo or in vitro to cells or tissues using standard transfection techniques well known in the art.

Other methods relating to the use of viral vectors in gene therapy can be found in, e.g., Kay, M. A., Chest 111(6 Supp.):138S-142S (1997); Ferry, N. and Heard, J. M., Hum. Gene Ther. 9:1975-81 (1998); Shiratory, Y. et al., Liver 19:265-74 (1999); Oka, K. et al., Curr. Opin. Lipidol. 11:179-86 (2000); Thule, P. M. and Liu, J. M., Gene Ther. 7:1744-52 (2000); Yang, N. S., Crit. Rev. Biotechnol. 12:335-56 (1992); Alt, M., J Hepatol. 23:746-58 (1995); Brody, S. L. and Crystal, R. G., Ann. N.Y. Acad. Sci. 716:90-101 (1994); Strayer, D. S., Expert Opin. Investig. Drugs 8:2159-2172 (1999); Smith-Arica, J. R. and Bartlett, J. S., Curr. Cardiol. Rep. 3:43-49 (2001); Lee, H. C. et al., Nature 408:483-8 (2000), U.S. Pat. No. 6,365,150,. U.S. Pat. No. 6,596,270, U.S. Pat. No. 6,573,092, U.S. Pat. No. 6,475,757, U.S. Pat. No. 6,465,253, U.S. Pat. No. 5,965,541, U.S. Pat. No. 6,635,476, U.S. Pat. No. 6,348,352, U.S. Pat. No. 6,312,681, U.S. Pat. No. 5,994,134, U.S. Pat. No. 5,932,210, U.S. Pat. No. 5,837,520, U.S. Pat. No. 6,689,600, U.S. Pat. No. 6,531,456, U.S. Pat. No. 6,416,992, U.S. Pat. No. 6,211,163, and U.S. Pat. No. 6,207,457.

Gene therapy vectors can be delivered to a subject, a mammal, preferably a human, by, for example, intravenous injection, local administration (see U.S. Pat. No. 5,328,470) or by stereotactic injection [see e.g., Chen et al., Proc. Natl. Acad. Sci. USA 91:3054-3057 (1994)]. The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The present invention also provides a pharmaceutical composition comprising an amount of the gene product of a claudin gene associated gene effective to reverse the cancerous phenotype of a cancer cell and a pharmaceutically acceptable carrier.

In another embodiment of the above-described methods, the cancer cell includes, but is not limited to, a breast, cervical, colon, prostate, nasopharyngeal, lung connective tissue and nervous system cells. The cancer cell further includes cells from glioblastoma multiforme, lymphomas and leukemia.

The nucleic acids or vectors containing nucleic acids encoding claudin-3, claudin-4 or claudin-9 are generally administered in a physiologically acceptable buffered solution that can be comprised of one or more components that promote sterility, stability and/or activity. Any means convenient for introducing the nucleic acid/vector preparation to a desired location with the body can be employed, including, for example, by intravenous or localized injection, by infusion from a catheter, intranasal delivery or by aerosol delivery.

The nucleic acids or vectors containing nucleic acids encoding claudin-3, claudin-4 or claudin-9 are administered in a therapeutically effective amount sufficient to promote the expression of claudin-3, claudin-4 or claudin-9 in the tumor cells causing the cells to form tight junctions to inhibit metastasis of a tumor. A therapeutically effective amount refers to an amount effective, at dosages and for periods of time necessary to achieve the desired therapeutic effect. The therapeutically effective amount may differ for different forms of cancer, age, sex or weight of an individual.

Diagnosing the Presence of Metastatic Cancer.

According to the present invention, metastatic neoplasm can be diagnosed by determining whether or not claudin-3, claudin-4 or claudin-9 are expressed in a tissue type that normally expresseses these proteins. There are a number of ways to determine this including the use of antibodies to detect the presence of the extracellular portion of the proteins or by determining the presence and amount mRNA coding for claudin-3, claudin-4 or claudin-9 in the cytoplasm of the cell. The methods of diagnosing cancer tissues of the invention are preferably performed using human cancer patient tumor samples, most preferably samples preserved, for example in paraffin, and prepared for histological and immunohistochemical analysis.

For the purposes of this invention, the term “tumor sample” is intended to include resected solid tumors, biopsy material, pathological specimens, bone marrow aspirates, and blood samples comprising neoplastic cells of hematopoietic origin, as well as benign tumors, particularly tumors of certain tissues such as brain and the central nervous system. One of ordinary skill will appreciate that samples derived from solid tumors will require combinations of physical and chemical/enzymatic disaggregation to separate neoplastic cells from stromal cells and infiltrating hematopoietic cells, while hematopoietic tumor samples including leukemias and lymphomas will be obtained as mixed cell populations in blood, serum or plasma, and will require separation from non-neoplastic components thereof, particularly from red blood cells that can be lysed by treatment with hypotonic solutions and from other nucleated cells, whereby separation is achieved by differential centrifugation and other methods known in the art.

Use of Immunological Reagents to Detect the Expresson of Claudin-3, Claudin-4 or Claudin-9

For the purposes of this invention, the term “immunological reagents” is intended to encompass antisera and antibodies, particularly monoclonal antibodies, as well as fragments thereof (including F(ab), F(ab).sub.2, F(ab)′ and F.sub.v fragments) that bind to claudin-3, claudin-4 or claudin-9. Also included in the definition of immunological reagent are chimeric antibodies, humanized antibodies, and recombinantly-produced antibodies and fragments thereof, as well as aptamers (i.e., oligonucleotides capable of interacting with target molecules such as peptides). Immunological methods used in conjunction with the reagents of the invention include direct and indirect (for example, sandwich-type) labeling techniques, immunoaffinity columns, immunomagnetic beads, fluorescence activated cell sorting (FACS), enzyme-linked immunosorbent assays (ELISA), and radioimmune assay (RIA), most preferably FACS. For use in these assays, the neoplastic immunological reagents can be labeled, using fluorescence, antigenic, radioisotopic or biotin labels, among others, or a labeled secondary or tertiary immunological detection reagent can be used to detect binding of the neoplastic immunological reagents (i.e., in secondary antibody (sandwich) assays) used in determing the presence of claudin-3, claudin-4 or claudin-9. Examples of immunological reagents useful in the practice of this invention include antibodies, most preferably monoclonal antibodies that recognize claudin-3, claudin-4 or claudin-9.

The immunological reagents of the invention are preferably detectably-labeled, most preferably using fluorescent labels that have excitation and emission wavelengths adapted for detection using commercially-available instruments such as and most preferably fluorescence activated cell sorters. Examples of fluorescent labels useful in the practice of the invention include phycoerythrin (PE), fluorescein isothiocyanate (FITC), rhodamine (RH), Texas Red (TX), Cy3, Hoechst 33258, and 4′,6-diamidino-2-phenylindole (DAPI). Such labels can be conjugated to immunological reagents, such as antibodies and most preferably monoclonal antibodies using standard techniques [Maino et al., Cytometry 20:127-133 (1995)].

Detection of Claudin-3. Claudin-4 and Claudin-9 Using Nucleic Acid Hybridization Techniques

The expression of claudin-3, claudin-4 and claudin-9 can be determined using nucleic acids and associated hybridization methods to detect the presence of mRNA within a cell of interest. For example, a nucleic acid that is complementary to and hybridizes under stringent conditions to the mRNA of a section of claudin-3, claudin-4 or claudin-9 can be detectably labeled. Such a detectably labeled nucleic acid molecule can be contacted with a cell or an extract of a cell to detect the presence and amount of the mRNA that encodes claudin-3, claudin-4 or claudin-9. The amount of nucleic acids that encode claudin-3, claudin-4 or claudin-9 correlates well with the expression of the claudins in a cell. The selection of an appropriate nucleic acid molecules for use as a probe can be made by studying the nucleic acid sequences of SEQ ID NOs: 1, 3 and Sand determining an appropriate length. A unique sequence should be determined that selectivey hybridizes under stringent conditions to the mRNA of claudin-3, claudin-4 or claudin-9.

The term “stringent conditions” as used herein refers to parameters with which the art is familiar. Nucleic acid hybridization parameters may be found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. More specifically, stringent conditions, as used herein, refers, for example, to hybridization at 65.degree. C. in hybridization buffer (3.5.times.SSC, 0.02% Ficoll, 0.02% polyvinyl pyrrolidone, 0.02% Bovine Serum Albumin, 2.5 mM NaH.sub.2 PO.sub.4 (pH7), 0.5% SDS, 2 mM EDTA). SSC is 0.15 M sodium chloride/0.015 M sodium citrate, pH7; SDS is sodium dodecyl sulphate; and EDTA is ethylenediaminetetracetic acid. After hybridization, the membrane upon which the DNA is transferred is washed, for example, in 2.times.SSC at room temperature and then at 0.1-0.5.times.SSC/0.1.times.SDS at temperatures up to 68.degree. C.

There are other conditions, reagents, and so forth which can be used, which result in a similar degree of stringency. The skilled artisan will be familiar with such conditions, and thus they are not given here. It will be understood, however, that the skilled artisan will be able to manipulate the conditions in a manner to permit the clear identification of homologs and alleles of cancer associated antigen nucleic acids of the invention (e.g., by using lower stringency conditions). The skilled artisan also is familiar with the methodology for screening cells and libraries for expression of such molecules which then are routinely isolated, followed by isolation of the pertinent nucleic acid molecule and sequencing.

A preferred method to detecting the claudin transcripts in genetic material derived from cells uses polymerase chain reaction (PCR) technology. PCR technology is practiced routinely by those having ordinary skill in the art and its uses in diagnostics are well known and accepted. Methods for practicing PCR technology are disclosed in “PCR Protocols: A Guide to Methods and Applications”, Innis, M. A., et al. Eds. Academic Press, Inc. San Diego, Calif. (1990). Applications of PCR technology are disclosed in “Polymerase Chain Reaction” Erlich, H. A., et al., Eds.

Cold Spring Harbor Press, Cold Spring Harbor, N.Y. (1989). U.S. Pat. No. 4,683,202, U.S. Pat. No. 4,683,195, U.S. Pat. No. 4,965,188 and U.S. Pat. Nos. 5,075,216 describe methods of performing PCR. PCR may be routinely practiced using Perkin Elmer Cetus GENE AMP RNA PCR kit, Part No. N808-0017.

PCR technology allows for the rapid generation of multiple copies of DNA sequences by providing 5′ and 3′ primers that hybridize to sequences present in an RNA or DNA molecule, and further providing free nucleotides and an enzyme which fills in the complementary bases to the nucleotide sequence between the primers with the free nucleotides to produce a complementary strand of DNA. The enzyme will fill in the complementary sequences adjacent to the primers. If both the 5′ primer and 3′ primer hybridize to nucleotide sequences on the same small fragment of nucleic acid, exponential amplification of a specific double-stranded size product results. If only a single primer hybridizes to the nucleic acid fragment, linear amplification produces single-stranded products of variable length.

PCR primers can be designed routinely by those having ordinary skill in the art using sequence information. The nucleotide sequences of claudin-3, claudin-4 and claudin-9 transcripts are set forth in SEQ ID NOs: 1, 3 and 5 respectively. To perform this method, RNA is extracted from cells in a sample and tested or used to make cDNA using well known methods and readily available starting materials.

Those having ordinary skill in the art can readily prepare PCR primers. A set of primers generally contains two primers. When performing PCR on extracted mRNA or cDNA generated therefrom, if the claudin transcript or cDNA generated thererefrom is present, multiple copies of the mRNA or cDNA will be made. If it is not present, PCR will not generate a discrete detectable product. Primers are generally 8-50 nucleotides, preferably about 15-35 nucleotides, more preferably 18-28 nucleotides, which are identical or complementary to and therefor hybridize to the CLAUDIN transcript or cDNA generated therefrom. In preferred embodiments, the primers are each 15-35 nucleotide, more preferably 18-28 nucleotide fragments of SEQ ID NOs: 1, 3 or 9. The primer must hybridize to the sequence to be amplified.

Typical primers are 18-28 nucleotides in length and are generally have 500 to 60% G+C composition. The entire primer is preferably complementary to the sequence to which it must hybridize. Preferably, primers generate PCR products 100 base pairs to 2000 base pairs. However, it is possible to generate products of 50 to up to 10 kb and more. If mRNA is used as a template, the primers must hybridize to mRNA sequences. If cDNA is used as a template, the primers must hybridize to cDNA sequences.

The mRNA or cDNA is combined with the primers, free nucleotides and enzyme following standard PCR protocols. The mixture undergoes a series of temperature changes. If the CLAUDIN transcript or cDNA generated therefrom is present, that is, if both primers hybridize to sequences on the same molecule, the molecule comprising the primers and the intervening complementary sequences will be exponentially amplified. The amplified DNA can be easily detected by a variety of well known means. If no CLAUDIN transcript or cDNA generated therefrom is present, no PCR product will be exponentially amplified. The PCR technology therefore provides an extremely easy, straightforward and reliable method of detecting the CLAUDIN transcript in a sample.

PCR product may be detected by several well known means. The preferred method for detecting the presence of amplified DNA is to separate the PCR reaction material by gel electrophoresis and stain the gel with ethidium bromide in order to visual the amplified DNA if present. A size standard of the expected size of the amplified DNA is preferably run on the gel as a control.

In some instances, such as when unusually small amounts of RNA are recovered and only small amounts of cDNA are generated therefrom, it is desirable or necessary to perform a PCR reaction on the first PCR reaction product. That is, if difficult to detect quantities of amplified DNA are produced by the first reaction, a second PCR can be performed to make multiple copies of DNA sequences of the first amplified DNA. A nested set of primers are used in the second PCR reaction. The nested set of primers hybridize to sequences downstream of the 5′ primer and upstream of the 3′ primer used in the first reaction.

The present invention includes oligonucleotide which are useful as primers for performing PCR methods to amplify the CLAUDIN transcript or cDNA generated therefrom.

According to the invention, diagnostic kits can be assembled which are useful to practice methods of detecting the presence of the CLAUDIN transcript or cDNA generated therefrom in non-colorectal samples. Such diagnostic kits comprise oligonucleotide which are useful as primers for performing PCR methods. It is preferred that diagnostic kits according to the present invention comprise a container comprising a size marker to be run as a standard on a gel used to detect the presence of amplified DNA. The size marker is the same size as the DNA generated by the primers in the presence of the the CLAUDIN transcript or cDNA generated therefrom. Additional components in some kits include instructions for carrying out the assay. Additionally the kit may optionally comprise depictions or photographs that represent the appearence of positive and negative results.

PCR assays are useful for detecting the CLAUDIN transcript in homogenized tissue samples and cells in body fluid samples. It is contemplated that PCR on the plasma portion of a fluid sample could be used to detect the CLAUDIN transcript.

Another method of determining whether a sample contains cells expressing CLAUDIN is by branched chain oligonucleotide hybridization analysis of mRNA extracted from a sample. Branched chain oligonucleotide hybridization may be performed as described in U.S. Pat. No. 5,597,909, U.S. Pat. No. 5,437,977 and U.S. Pat. No. 5,430,138, which are each incorporated herein by reference. Reagents may be designed following the teachings of those patents and that sequence of the CLAUDIN transcript.

Another method of determining whether a sample contains cells expressing CLAUDIN is by Northern Blot analysis of mRNA extracted from a non-colorectal sample. The techniques for performing Northern blot analyses are well known by those having ordinary skill in the art and are described in Sambrook, J. et al., (1989) Molecular Cloning: A Laboratory Manual, Cold Spring Harbor Laboratory Press, Cold Spring Harbor, N.Y. mRNA extraction, electrophoretic separation of the mRNA, blotting, probe preparation and hybridization are all well known techniques that can be routinely performed using readily available starting material.

The mRNA is extracted using poly dT columns and the material is separated by electrophoresis and, for example, transferred to nitrocellulose paper. Labelled probes made from an isolated specific fragment or fragments can be used to visualize the presence of a complementary fragment fixed to the paper. Probes useful to identify mRNA in a Northern Blot have a nucleotide sequence that is complementary to the CLAUDIN transcript. Those having ordinary skill in the art could use the sequence information in SEQ ID NOs: 1, 3 or 5 to design such probes or to isolate and clone the the CLAUDIN transcript or cDNA generated therefrom to be used as a probe. Such probes are at least 15 nucleotides, preferably 30-200, more preferably 40-100 nucleotide fragments and may be the entire CLAUDIN transcript.

According to the invention, diagnostic kits can be assembled which are useful to practice methods of detecting the presence of the CLAUDIN transcript in non-colorectal samples by Northern blot analysis. Such diagnostic kits comprise oligonucleotide which are useful as probes for hybridizing to the mRNA. The probes may be radiolabelled. It is preferred that diagnostic kits according to the present invention comprise a container comprising a size marker to be run as a standard on a gel. It is preferred that diagnostic kits according to the present invention comprise a container comprising a positive control which will hybridize to the probe. Additional components in some kits include instructions for carrying out the assay. Additionally the kit may optionally comprise depictions or photographs that represent the appearence of positive and negative results.

Northern blot analysis is useful for detecting the CLAUDIN transcript in homogenized tissue samples and cells in body fluid samples. It is contemplated that PCR on the plasma portion of a fluid sample could be used to detect the CLAUDIN transcript.

Another method of detecting the presence of the CLAUDIN transcript by oligonucleotide hybridization technology. Oligonucleotide hybridization technology is well known to those having ordinary skill in the art. Briefly, detectable probes which contain a specific nucleotide sequence that will hybridize to nucleotide sequence of the CLAUDIN transcript. RNA or cDNA made from RNA from a sample is fixed, usually to filter paper or the like. The probes are added and maintained under conditions that permit hybridization only if the probes fully complement the fixed genetic material. The conditions are sufficiently stringent to wash off probes in which only a portion of the probe hybridizes to the fixed material. Detection of the probe on the washed filter indicate complementary sequences.

Probes useful in oligonucleotide assays at least 18 nucleotides of complementary DNA and may be as large as a complete complementary sequence to the CLAUDIN transcript. In some preferred embodiments the probes of the invention are 30-200 nucleotides, preferably 40-100 nucleotides.

One having ordinary skill in the art, using the sequence information disclosed in SEQ ID NOs: 1, 3 or 5 can design probes which are fully complementary to the CLAUDIN transcript. Hybridization conditions can be routinely optimized to minimize background signal by non-fully complementary hybridization. In some preferred embodiments, the probes are full length clones. Probes are at least 15 nucleotides, preferably 30-200, more preferably 40-100 nucleotide fragments and may be the entire CLAUDIN transcript.

The present invention includes labelled oligonucleotide which are useful as probes for performing oligonucleotide hybridization. That is, they are fully complementary with the CLAUDIN transcript. For example, the mRNA sequence includes portions encoded by different exons. The labelled probes of the present invention are labelled with radiolabelled nucleotides or are otherwise detectable by readily available nonradioactive detection systems.

According to the invention, diagnostic kits can be assembled which are useful to practice oligonucleotide hybridization methods of the invention. Such diagnostic kits comprise a labelled oligonucleotide which encodes portions of the CLAUDIN transcript. It is preferred that labelled probes of the oligonucleotide diagnostic kits according to the present invention are labelled with a radionucleotide. The oligonucleotide hybridization-based diagnostic kits according to the invention preferably comprise DNA samples that represent positive and negative controls. A positive control DNA sample is one that comprises a nucleic acid molecule which has a nucleotide sequence that is fully complementary to the probes of the kit such that the probes will hybridize to the molecule under assay conditions. A negative control DNA sample is one that comprises at least one nucleic acid molecule, the nucleotide sequence of which is partially complementary to the sequences of the probe of the kit. Under assay conditions, the probe will not hybridize to the negative control DNA sample. Additional components in some kits include instructions for carrying out the assay. Additionally the kit may optionally comprise depictions or photographs that represent the appearence of positive and negative results.

Oligonucleotide hybridization techniques are useful for detecting the CLAUDIN transcript in homogenized tissue samples and cells in body fluid samples. It is contemplated that PCR on the plasma portion of a fluid sample could be used to detect the CLAUDIN transcript.

The present invention relates to in vitro kits for evaluating tissues samples to determine the level of metastasis and to reagents and compositions useful to practice the same.

These techniques for determining the presence of mRNA of a polypeptide have resulted in the production of various microarrays, bioarray, biochips and biochip arrays. As used herein, the terms “microarray,” “bioarray,” “biochip” and “biochip array” refer to an ordered spatial arrangement of immobilized biomolecular probes arrayed on a solid supporting substrate. Preferably, the biomolecular probes are immobilized on second linker moieties in contact with polymeric beads, wherein the polymeric beads are immobilized on first linker moieties in contact with the solid supporting substrate. Biochips, as used in the art, encompass substrates containing arrays or microarrays, preferably ordered arrays and most preferably ordered, addressable arrays, of biological molecules that comprise one member of a biological binding pair. Typically, such arrays are oligonucleotide arrays comprising a nucleotide sequence that is complementary to at least one sequence of a nucleic acid that encodes claudin-3, claudin-4 or claudin-9. Alternatively, and preferably, proteins, peptides or other small molecules can be arrayed in such biochips for performing, inter alia, immunological analyses (wherein the arrayed molecules are antigens) or assaying biological receptors (wherein the arrayed molecules are ligands, agonists or antagonists of said receptors). Useful microarrays for detecting differential gene expression are described, inter alia, in U.S. Pat. No. 6,040,138 to Lockhart et al. (commercially-available from Affymetrix, Inc., Santa Clara, Calif.) and U.S. Pat. No. 6,004,755 to Wang (commercially-available from Incyte Inc., Palo Alto, Calif.) and are also commercially available, inter alia, from Research Genetics (Huntsville, Ala.).

Gene expression analysis is performed to detect differences in gene expression between populations of neoplastic, metastatic cells and normal cells to determine whether or not claudin-3, claudin-4 or claudin-9 are being expressed. Hybridization of gene expression microarrays produces pattern of gene expression of claudin-3, claudin-4 or claudin-9. Identification of genes and patterns of genes differentially expressed in these cells is established by comparison of the gene expression pattern obtained by performing the microarray hybridization analysis on cDNA from neoplastic cells in comparison to that of normal tissue.

This invention will be better understood from the experimental details, which follow. However, one skilled in the art will readily appreciate that the specific methods and results discussed are merely illustrative of the invention as described more fully in the claims, which follow thereafter.

### EXAMPLE 1

Tight Junction Gene Expression in Respiratory Epithelium: Differential mRNA Expression of Claudin, Occludin, Zonulin and JAM

Claudins, Junctional Adhesion Molecule (JAM) and Zonulin are multigene superfamily proteins and components of the Tight Junction (TJ). They are expressed in all epithelia and aid the TJ in polarizing cells and serve a barrier function. Discovering the role these proteins play in the composition and function of the TJ from respiratory epithelial tissues is critical to exploiting the TJ for tissue permeability of therapeutic agents. Here we report results from our analysis of the TJ gene expression in normal and immortalized respiratory epithelium by RT-PCR. Specific primers to each of several claudins (CLDN 1-12 and 14-20) were designed using the Primer3 program. mRNA was extracted from differentiated and undifferentiated primary epithelial cells (EpiAirway, MatTek Inc.) and semi-quantitative RT-PCR performed. SEQ ID NOs: 7 and 8 were the primers used to amplify the cDNA encoding claudin-3. SEQ ID NOs: 9 and 10 were the primers used to amplify the cDNA encoding claudin-4. SEQ ID NOs: 11 and 12 were the primers used to amplify the cDNA encoding claudin-9. Products from these reactions were analyzed by EtBr-stained agarose gel electrophoresis and densitometry. In normal epithelium, CLDN 1, 3, 4, 9, 12 and 20 showed high level RNA expression, while CLDN 5, 7, 10, 11, 14 and 16 were at a much lower level. mRNA levels of the other claudins were undetectable. Of the high-level claudin expression group, CLDN 1, 12 and 20 were found in both differentiated and undifferentiated tissues. However, only CLDN 3, 4 and 9 were expressed in differentiated tissues. Levels of other TJ transcripts (JAM-1, Occludin, ZO-1, ZO-2 and ZO-3) showed no differential expression. We also report expression profiles in primary respiratory cells (16HBE14o-) and RPMI 2650, a cell line which does not display TJs. These results demonstrate the differential expression pattern of TJ proteins in airway epithelium and assist in focusing efforts to create TJ modulators as pharmaceutical targets to promote paracellular drug delivery.

The teachings of all of the references cited herein are incorporated in their entirety by reference.

What is claimed is:

1. A method for treating cancer in a mammal comprised of administering a therapeutically effective amount of an agent that induces the expression of claudin-3, claudin-4 or claudin-9 within a cancerous cell.

2. The method of claim 1 wherein a nucleic acid that encodes a claudin is administered to the mammal under conditions wherein the nucleic acid is transfected into the cancerous cell and the claudin is produced in the cell.

3. The method of claim 2 wherein the nucleic acid is contained within a viral vector.

4. The method of claim 3 wherein the viral vector selected from the group consisting of adenoviral vector, retroviral vector and adenovirus-associated virus vector.

5. The method of claim 1 wherein the mammal is a human.

6. A method for inhibiting metastasis of cancerous tissue, comprising the step of administering a therapeutically effective amount of an agent that induces the expression of claudin-3, claudin-4 or claudin-9 within the cancerous tissue.

7. The method of claim 6 wherein one or more nucleic acids that encode claudin-3, claudin-4 or claudin-9 is administered to the mammal under conditions wherein the nucleic acid is transfected into the cancerous cell and the claudin is produced in the cell.

8. The method of claim 7 wherein the nucleic acid contained within a viral vector.

9. The method of claim 6 wherein the viral vector is selected from the group consisting of adenoviral vector, retroviral vector, lentivirus and adenovirus-associated virus vector.

10. The method of claim 6 wherein the mammal is a human.

11. A method of diagnosis of neoplasia in a mammal, which method comprises analyzing the expression of the expression of claudins 3, 4 and 9 within a tissue, wherein the tissue is metastatic cancer, if one or more of the claudins are underexpressed.

12. The method of claim 11 wherein the expression of claudin-3, claudin 4 and claudin-9 are carried out by a nucleic acid hybridization technique.

13. An in vitro method of determining whether or not an individual has metastasized cancer cells comprising the steps of examining a sample of tissue and/or body fluids from an individual to determine whether claudin-3, claudin-4 or claudin-9 transcripts are being expressed by cells in said sample wherein under expression of said claudin transcripts is indicative of the presence of metastasized colorectal cancer cells in said sample.

14. The method of claim 13 wherein expression of said claudin transcripts by said cells is determined by polymerase chain reaction wherein said sample is contacted with primers that selectively amplify a claudin mRNA transcript or cDNA generated therefrom.

15. An in vitro method of determining whether or not an individual has metastasized cancer comprising the steps of examining a sample of tissue from an individual to determine whether a claudin-3, claudin-4 or claudin-9 mRNA transcript is expressed in said sample, wherein the underexpression of said claudin mRNA transcript in said sample indicates that said individual has metastasized cancer.

16. The method of claim 15 wherein said claudin mRNA transcript is detected by polymerase chain reaction assay using primers which amplify claudin-3, claudin-4 or claudin-9 transcript sequences.

17. An in vitro PCR assay kit for determining whether or not an individual has metastatic cancer by detecting the expression of claudin-3, claudin-4 or claudin-9 mRNA transcript in a sample of tissue and/or body fluids from an individual, wherein the underexpression of said claudin transcript in said sample indicates that individual has metastatic cancer, said kit comprising:
a first container comprising PCR primers that amplify said claudin transcript or cDNA generated therefrom; and
a second container comprising a nucleic acid marker, said marker being labeled and being able to hybridize to said transcript of cDNA.

18. A kit for testing a mammal for the presence or a metastatic, said kit comprising a antibody or antibody fragment that specifically binds to a mammalian a claudin-3, claudin-4 or claudin-9 polypeptide

19. The kit of claim 18, said kit further comprising a means for detecting said binding of said antibody or antibody fragment to said claudin polypeptide.

20. The kit of claim 18, wherein said mammal is a human.

21. The kit of claim 18, wherein said antibody is a rodent antibody.

22. The kit of claim 18, wherein said antibody is a polyclonal antibody.

23. The kit of claim 18, wherein said antibody is a monoclonal antibody.

24. The kit of claim 18 wherein said is an enzyme-linked immunosorbent assay for determining the presence of claudin-3, claudin-4 or claudin-9

25. The kit of claim 18 wherein said kit is a radioimmunoassay.

26. The kit of claim 18 wherein said kit is a fluorescent immunoassay employing an fluoroescent antibody or antibody fragment that binds to claudin-3, claudin-4 or claudin-9.

27. A method for determining whether or not a cell is a metastatic cancerous cell comprising bringing the cell into contact with an antibody or antibody fragment or other protein or polypeptide that binds to claudin-3, claudin-4 or claudin-9; and detecting whether or not the antibody or antibody fragment binds to the cell, wherein if the antibody or antibody fragment does not bind to the cell, then the cell is a metastatic cancerous cell.

28. The method of claim 27, wherein said method is an enzyme-linked immunosorbent assay for determining the presence of claudin-3, claudin-4 or claudin-9

29. The method of claim 27 wherein said method is a radioimmunoassay.

30. The method of claim 27 wherein said method is a fluorescent immunoassay employing an fluoroescent antibody or antibody fragment that binds to claudin-3, claudin-4 or claudin-9.


SEQUENCE LISTING (12 sequences)

SEQ ID NO: 1 - DNA, 1294 bp, Homo sapiens
  CDS (198)...(860)
caaagccacaggcaggtgcaggcgcagccgcggcgagagcgtatggagccgagccgttagcgcgcgccgtcggtgagtcagtccgtccgtccgtccgtccgtcggggcgccgcagctcccgccaggcccagcggccccggcccctcgtctccccgcacccggagccacccggtggagcgggccttgccgcggcagccatgtccatgggcctggagatcacgggcaccgcgctggccgtgctgggctggctgggcaccatcgtgtgctgcgcgttgcccatgtggcgcgtgtcggccttcatcggcagcaacatcatcacgtcgcagaacatctgggagggcctgtggatgaactgcgtggtgcagagcaccggccagatgcagtgcaaggtgtacgactcgctgctggcactgccacaggaccttcaggcggcccgcgccctcatcgtggtggccatcctgctggccgccttcgggctgctagtggcgctggtgggcgcccagtgcaccaactgcgtgcaggacgacacggccaaggccaagatcaccatcgtggcaggcgtgctgttccttctcgccgccctgctcaccctcgtgccggtgtcctggtcggccaacaccattatccgggacttctacaaccccgtggtgcccgaggcgcagaagcgcgagatgggcgcgggcctgtacgtgggctgggcggccgcggcgctgcagctgctggggggcgcgctgctctgctgctcgtgtcccccacgcgagaagaagtacacggccaccaaggtcgtctactccgcgccgcgctccaccggcccgggagccagcctgggcacaggctacgaccgcaaggactacgtctaagggacagacgcagggagaccccaccaccaccaccaccaccaacaccaccaccaccaccgcgagctggagcgcgcaccaggccatccagcgtgcagccttgcctcggaggccagcccacccccagaagccaggaagcccccgcgctggactggggcagcttccccagcagccacggctttgcgggccgggcagtcgacttcggggcccagggaccaacctgcatggactgtgaaacctcacccttctggagcacggggcctgggtgaccgccaatacttgaccaccccgtcgagccccatcgggccgctgcccccatgctcgcgctgggcagggaccggcagccctggaaggggcacttgatatttttcaataaaagcctttcgttttgcaaaaaaaaaaaaaaaaaaaaaaaaaaaaaaaaaaaaaaaaaaaaa

SEQ ID NO: 2 - protein, 220 aa, Homo sapiens
MSMGLEITGTALAVLGWLGTIVCCALPMWRVSAFIGSNIITSQNIWEGLWMNCVVQSTGQMQCKVYDSLLALPQDLQAARALIVVAILLAAFGLLVALVGAQCTNCVQDDTAKAKITIVAGVLFLLAALLTLVPVSWSANTIIRDFYNPVVPEAQKREMGAGLYVGWAAAALQLLGGALLCCSCPPREKKYTATKVVYSAPRSTGPGASLGTGYDRKDYV

SEQ ID NO: 3 - DNA, 1712 bp, Homo sapiens
  CDS (206)...(835)
ggcacgaggggcagctgtcggctggaaggaactggtctgctcacacttgctggcttgcgcatcaggactggctttatctcctgactcacggtgcaaaggtgcactctgcgaacgttaagtccgtccccagcgcttggaatcctacggcccccacagccggatcccctcagccttccaggtcctcaactcccgcggacgctgaacaatggcctccatggggctacaggtaatgggcatcgcgctggccgtcctgggctggctggccgtcatgctgtgctgcgcgctgcccatgtggcgcgtgacggccttcatcggcagcaacattgtcacctcgcagaccatctgggagggcctatggatgaactgcgtggtgcagagcaccggccagatgcagtgcaaggtgtacgactcgctgctggcactgccgcaggacctgcaggcggcccgcgccctcgtcatcatcagcatcatcgtggctgctctgggcgtgctgctgtccgtggtggggggcaagtgtaccaactgcctggaggatgaaagcgccaaggccaagaccatgatcgtggcgggcgtggtgttcctgttggccggccttatggtgatagtgccggtgtcctggacggcccacaacatcatccaagacttctacaatccgctggtggcctccgggcagaagcgggagatgggtgcctcgctctacgtcggctgggccgcctccggcctgctgctccttggcggggggctgctttgctgcaactgtccaccccgcacagacaagccttactccgccaagtattctgctgcccgctctgctgctgccagcaactacgtgtaaggtgccacggctccactctgttcctctctgctttgttcttccctggactgagctcagcgcaggctgtgaccccaggagggccctgccacgggccactggctgctggggactggggactgggcagagactgagccaggcaggaaggcagcagccttcagcctctctggcccactcggacaacttcccaaggccgcctcctgctagcaagaacagagtccaccctcctctggatattggggagggacggaagtgacagggtgtggtggtggagtggggagctggcttctgctggccaggatggcttaaccctgactttgggatctgcctgcatcggtgttggccactgtccccatttacattttccccactctgtctgcctgcatctcctctgttgcgggtaggccttgatatcacctctgggactgtgccttgctcaccgaaacccgcgcccaggagtatggctgaggccttgcccacccacctgcctgggaagtgcagagtggatggacgggtttagaggggaggggcgaaggtgctgtaaacaggtttgggcagtggtgggggagggggccagagaggcggctcaggttgcccagctctgtggcctcaggactctctgcctcacccgcttcagcccagggcccctggagactgatcccctctgagtcctctgccccttccaaggacactaatgagcctgggagggtggcagggaggaggggacagcttcacccttggaagtcctggggtttttcctcttccttctttgtggtttctgttttgtaatttaagaagagctattcatcactgtaattattattattttctacaataaatgggacctgtgcacaggaaaaaaaaaaaaaaaaaaaaaaaa

SEQ ID NO: 4 - protein, 209 aa, Homo sapiens
MASMGLQVMGIALAVLGWLAVMLCCALPMWRVTAFIGSNIVTSQTIWEGLWMNCVVQSTGQMQCKVYDSLLALPQDLQAARALVIISIIVAALGVLLSVVGGKCTNCLEDESAKAKTMIVAGVVFLLAGLMVIVPVSWTAHNIIQDFYNPLVASGQKREMGASLYVGWAASGLLLLGGGLLCCNCPPRTDKPYSAKYSAARSAAASNYV

SEQ ID NO: 5 - DNA, 1167 bp, Homo sapiens
  CDS (32)...(685)
tggggctgagaagacctaaccgaggggccagatggcttcgaccggcttagaactgctgggcatgaccctggctgtgctgggctggctggggaccctggtgtcctgcgccctgcccctgtggaaggtgaccgccttcatcggcaacagcatcgtggtggcccaggtggtgtgggagggcctgtggatgtcctgcgtggtgcagagcacgggccagatgcagtgcaaggtgtacgactcactgctggctctgccgcaggacctgcaggccgcacgtgccctctgtgtcattgccctcctgctggccctgcttggcctcctggtggccatcacaggtgcccagtgtaccacgtgtgtggaggacgaaggtgccaaggcccgtatcgtgctcaccgcgggggtcatcctcctcctcgccggcatcctggtgctcatccctgtgtgctggacggcgcacgccatcatccaggacttctacaaccccctggtggctgaggccctcaagcgggagctgggggcctccctctacctgggctgggcggcggctgcactgcttatgctgggcggggggctcctctgctgcacgtgccccccgccccaggtcgagcggccccgcggacctcggctgggctactccatcccctcccgctcgggtgcatctggactggacaagagggactacgtgtgaggcggaggtttcccctgggagcccactgctccccactgccccgccctttcgaccttggcctgatgaccagatgccctgctccatcacaacctccttccccaggaaaacccactttccaaaagcccaagctacacctggctgcagggctgggtcagctggcctggctgagctcttctcagtggggtcccctttgatgttctcccccaagttgggcagcctagaggtgttgggaaccctggcctgcccccacctccccagtaattgtttccttccgttgcccaggacactggctggccttccttctcttctgagccctcccctgccccaggaaccctggcctcaccaaaacagcagcagctcgttggctccaaaaccagggagcagaccatgccctcccaaccctggagttgtcagggagggcctgcccatcacctccctctccccaacatccccaccctcgagttggaaataaagagcatttg

SEQ ID NO: 6 - protein, 217 aa, Homo sapiens
MASTGLELLGMTLAVLGWLGTLVSCALPLWKVTAFIGNSIVVAQVVWEGLWMSCVVQSTGQMQCKVYDSLLALPQDLQAARALCVIALLLALLGLLVAITGAQCTTCVEDEGAKARIVLTAGVILLLAGILVLIPVCWTAHAIIQDFYNPLVAEALKRELGASLYLGWAAAALLMLGGGLLCCTCPPPQVERPRGPRLGYSIPSRSGASGLDKRDYV

SEQ ID NO: 7 - DNA, 19 bp, Homo sapiens
aaggtgtacgactcgctgc

SEQ ID NO: 8 - DNA, 20 bp, Homo sapiens
agtcccggataatggtgttg

SEQ ID NO: 9 - DNA, 19 bp, Homo sapiens
gacttctacaatccgctgg

SEQ ID NO: 10 - DNA, 20 bp, Homo sapiens
agcagagaggaacagagtgg

SEQ ID NO: 11 - DNA, 23 bp, Homo sapiens
catcatccaggacttctacaacc

SEQ ID NO: 12 - DNA, 22 bp, Homo sapiens
acgtagtccctcttgtccagtc